(19) United States
(12) Patent Application Publication
(10) Pub. No.: US 2006/0074278 A1
(43) Pub. Date: Apr. 6, 2006

(54) Tissue retractor producing a widened operating channel

(76) Inventors: Dominique Petit, Verton (FR); Vincent Sene, Cachan (FR)

Correspondence Address: BACHMAN & LAPOINTE, P.C., 900 CHAPEL STREET, SUITE 1201, NEW HAVEN, CT, 06510, US

(21) Appl. No.: 11/219,158

(22) Filed: Sep. 1, 2005

(30) Foreign Application Priority Data
Sep. 2, 2004 (FR) 0409300

(51) Int. Cl. A61B 1/32

(52) U.S. Cl. 600/224

(57) ABSTRACT

The present invention relates to a retractor for retracting a patient's tissues, of the type comprising two blades each exhibiting a proximal end and a distal end, said blades being arranged in such a way as to form an operating channel open at the proximal ends and at the distal ends of said blades, wherein the retractor comprises at least one additional blade to form an at least three-bladed retractor, said blades being parted from one another by pivoting of their distal ends so as to form a widened operating channel of conical shape.

The present invention relates to the field of the retraction and parting of soft tissue in order to form an operating field so that surgical procedures can be performed on a patient.

The present invention relates more particularly to a retractor for retracting a patient's tissues, of the type comprising blades each exhibiting a proximal end and a distal end. Said blades of the retractor are arranged in such a way as to form an operating channel open at the proximal ends and at the distal ends of said blades and which can be widened at the distal ends of said blades.

Such retractors are already known in the prior art.

In particular, international patent application WO 2004/002323 proposes a retractor comprising a working channel formed by a first portion coupled to a second portion. Said first and second portions can be moved one relative to the other from a non-elongated configuration to an elongated configuration so that the dimensions of the working channel are increased along its length, while at the same time minimizing the trauma caused to the skin and to the tissue.

According to one particular configuration of the invention, these portions pivot one with respect to the other in such a way as to offer a widened working channel. To do that, the proximal end of the retractor is equipped with extensions that are inclined with respect to the horizontal plane, on which extensions engagement members intended to collaborate with a parting instrument are arranged. The working channel is therefore widened by applying a lateral parting force to said engagement members.

Such a retractor does, however, have the disadvantage of allowing the working channel to be opened up in just two directions in space. It therefore follows, in order to widen the working channel in all directions, that a second retractor needs to be placed in the incision at right angles to the retractor initially inserted, something which, in the case of the retractor in the aforementioned international patent application, is still relatively difficult to do. Furthermore, positioning a second retractor at right angles to the first would, through necessity, limit the visibility of the working channel at the surface.

European patent application EP0455282 also discloses a surgical retractor intended in particular for cholecystectomy.

The retractor in particular comprises a retractor frame formed of a series of sides connected together by articulations to form an articulated polygonal frame, and a plurality of dilators fixed to the sides on the retractor frame, said dilators being of a more or less thin plate form and extending along an axis that intersects the retractor frame.

Because of the articulations used between each of the sides, the retractor, like the retractor described previously, has the disadvantage of allowing the flesh to be retracted in just two directions in space.

European patent application EP0246086 also relates to a retractor for laparoscopic cholecystectomies.

The retractor comprises, in particular, a base element comprising an opening of the window type for the passage of the surgical implements, and at least one pair of branches of tubular shape.

Now, because of their configuration, the branches, when parted, offer a working channel which is insufficient to allow surgical procedures to be performed in complete safety.

Furthermore, such a retractor proves to be somewhat difficult to use.

The present invention intends to remedy the disadvantages of the prior art by proposing a retractor that can be inserted into a patient's tissues via a minimal incision at the surface, while at the same time allowing the tissues to be retracted, deep within the incision, as much as possible so as to offer an operating field that is widened in all three directions in space.

Another object of the present invention is to provide a retractor that is simple to use, easy to handle and not very bulky.

Another object of the invention is to provide a retractor allowing precise adjustment of the extent of the operating field tailored to the surgical procedures.

To do this, the present invention relates to a retractor for retracting a patient's tissues, of the type comprising two blades each exhibiting a proximal end and a distal end, said blades being arranged in such a way as to form an operating channel open at the proximal ends and at the distal ends of said blades, and it is notable in that the retractor comprises at least one additional blade to form an at least three-bladed retractor, said blades being parted from one another by pivoting of their distal ends so as to form a widened operating channel of conical shape.

Thus, the retractor according to the invention allows the tissues to be retracted in all three directions in space, unlike the retractors of the prior art which allow the tissues to be distracted in just two directions.

According to a preferred embodiment of the invention, the retractor comprises a device for parting the blades which device consists of at least two bearing elements allowing a force to be exerted on the proximal end of said blades and thus part them.

Advantageously, the bearing elements constitute elements for adjusting the extent of separation of said blades.

Advantageously, the bearing elements consist of screws, each of said screws passing through a threaded orifice formed in said blades.

According to one advantageous embodiment of the invention, the retractor further comprises a tubular protective membrane surrounding said branches.

Advantageously, the blades have retaining notches for keeping the membrane fixed around said blades.

Advantageously, the membrane is elastic.

Advantageously, the membrane is made of tissue or of polymer, of the polyurethane type.

Advantageously, the blades are removable.

Advantageously, the blades are made of a radiotransparent material.

Advantageously, the retractor comprises at least one access orifice providing access to the space situated between the tissues and the blades or, when the retractor comprises a protective membrane as described hereinabove, access to the space situated between the membrane and the blades surrounded by the said membrane. The retractor, thus equipped with such an orifice, allows an implement, such as a suction implement or an implement delivering cold light, to pass. The implement, then positioned between the tissues and the blades or, when the retractor comprises a membrane, between said membrane and the blades, does not then block the working channel.

Advantageously, the retractor has a proximal part of flared shape, so as to allow good sight of the working channel.

Advantageously, the retractor comprises means of attachment to a support, of the table type, thus allowing the retractor to be stabilized once it has been positioned in the incision formed in the patient's tissues.

The present invention also relates to a retractor for retracting a patient's tissues, of the type comprising two blades each exhibiting a proximal end and a distal end, said blades being arranged in such a way as to form an operating channel open at the proximal ends and at the distal ends of said blades, wherein the retractor comprises a tubular protective membrane surrounding said blades.

Such a membrane makes it possible to prevent the retracted tissues from entering the spaces formed between the branches when these branches are in the parted position, so that the operating channel offers an optimum operating field.

As a preference, the retractor comprises at least one additional blade to form an at least three-bladed retractor, said blades being surrounded by the membrane.

Advantageously, the blades have retaining notches for keeping the membrane fixed around said blades.

Advantageously, the membrane is elastic.

Advantageously, the membrane is made of tissue or of polymer, and preferably of polyurethane.

Advantageously, said retractor will also comprise the technical characteristics of the retractor described previously (parting device, removable blades, blades made of a radiotransparent material, access orifice, flared proximal shape, means of attachment to a support).

The invention will be better understood with the aid of the description, given hereinafter purely by way of explanation, of one embodiment of the invention, with reference to the attached figures in which:

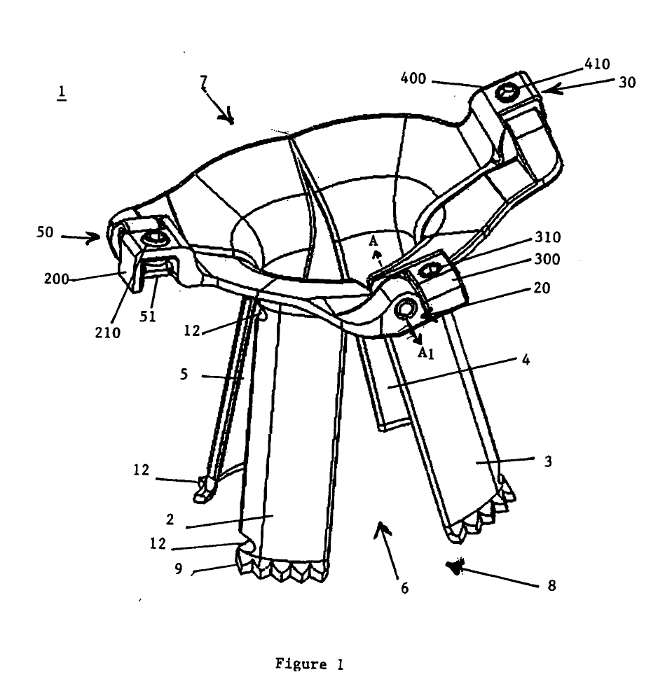

FIG. 1 illustrates a perspective view of a tissue tractor according to the invention when the retractor is in the parted position;

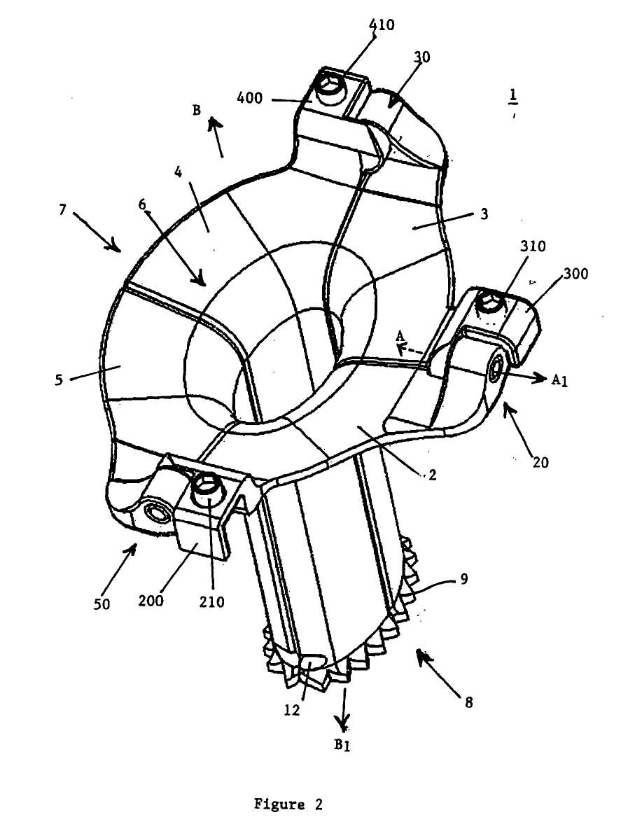

FIG. 2 illustrates a perspective view of the retractor of FIG. 1 in the contracted position; and

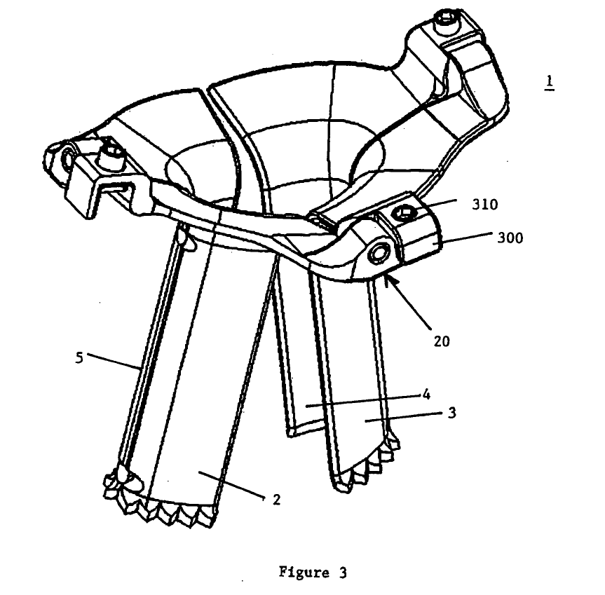

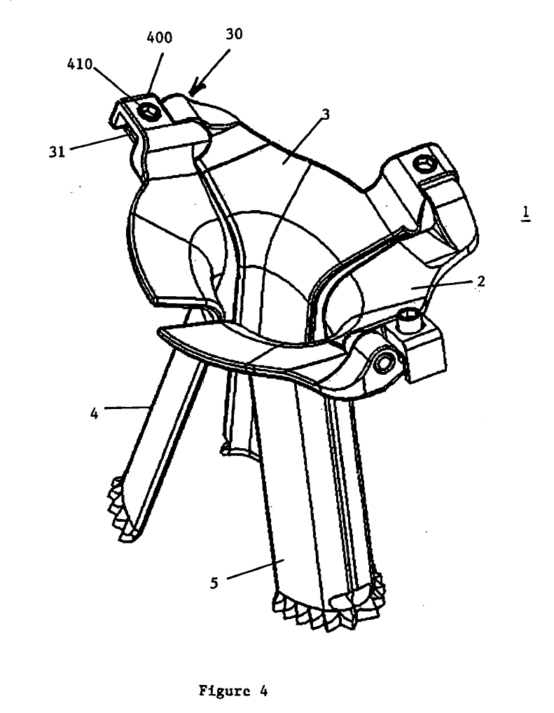

FIGS. 3 and 4 illustrate the retractor of FIGS. 1 and 2 in intermediate parted positions;

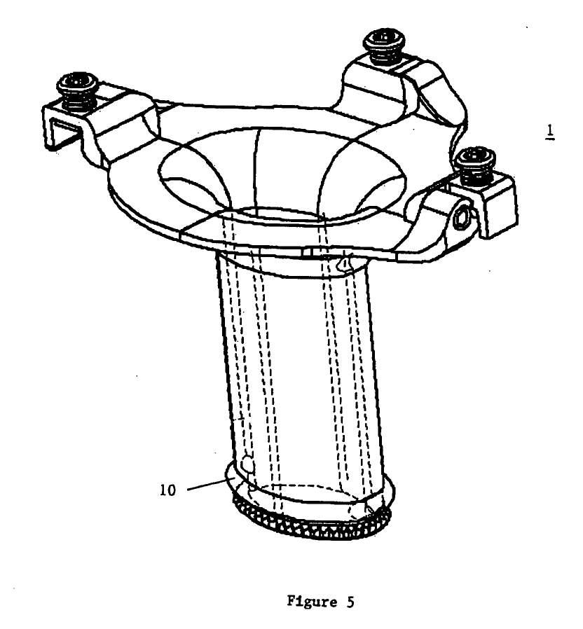

FIG. 5 illustrates a perspective view of the retractor of FIG. 1 equipped with a protective membrane, in the contracted position; and

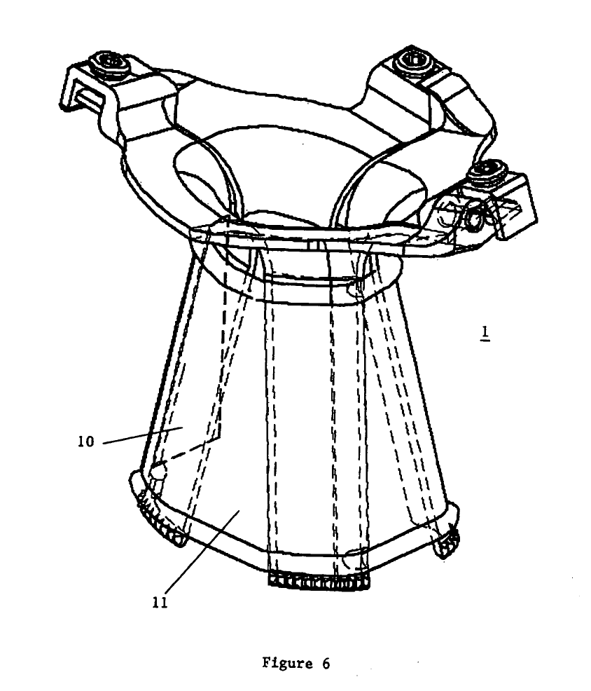

FIG. 6 illustrates a perspective view of the retractor of FIG. 5 in the parted position.

As previously explained, a tissue retractor is a piece of equipment that allows surgical procedures to be performed on a patient.

FIG. 1 illustrates a tissue retractor (1) according to the invention when this retractor is in a parted position.

In the example illustrated, the retractor (1) has four blades (2 to 5), each of the blades (2 to 5) having a respective proximal end and distal end.

Said blades (2 to 5) are arranged with respect to one another in such a way as to form an operating channel (6) open at its ends, the proximal ends and the distal ends of each of the blades (2 to 5) respectively constituting the proximal end (7) and the distal end (8) of said retractor (1).

Each of the blades (2 to 5) is connected to at least one adjacent blade at their respective proximal ends using a pivot connection (20, 30, 50) exhibiting a pivot axis AA1 perpendicular to the longitudinal axis BB1 of the retractor (1). A longitudinal axis is to be understood as meaning the longitudinal axis of the retractor (1) when the retractor is in the contracted position.

Thus, the blade (5) is connected to the blade (2) by the pivot connection (50), the blade (2) to the blade (3) by the pivot connection (20), and the blade (3) to the blade (4) by the connection (40). By contrast, the blades (4, 5) are not fixed, these already being fixed to the blades (3, 2) respectively.

The proximal end (7) of said retractor (1) is equipped with three retaining elements (200, 300, 400) intended respectively to accept an extension of the pivot connections (20, 30, 50), each extension butting up against one of said retaining elements (200, 300, 400).

Furthermore, said retaining elements (200, 300, 400) allow any translational movement with respect to the axis of pivoting AA1 of the pivot connections (20, 30, 50) to be prevented, and thus prevent the possibility of the retractor blades becoming disassembled.

Advantageously, the retaining elements (200, 300, 400), in the shape of an inverted U, are arranged on the proximal end of the blade adjacent to the blade bearing the pivot connection, the extension of the pivot connection extending toward said corresponding retaining element. Thus, the retaining elements (200, 300, 400) are borne on the proximal ends of the blades (20, 30, 40).

Each blade (2 to 5) of the retractor (1) thus connected may be parted independently of the adjacent blades by a pivoting movement toward the tissues, then returned to the “authorized” contracted position.

The parting of the four blades (2 to 5) of the retractor (1) is obtained by applying pressure to the extension of each of the pivot connections (20, 30, 50). Advantageously, the pressure exerted is applied by means of a bearing element (210, 310, 410), of the screw type, which passes through a threaded orifice in the corresponding retaining element so as to be in contact with the extension of the associated pivot connection.

Thus, when the set screws (210, 310, 410) are screwed into the retaining elements (200, 300, 400) as illustrated in FIG. 1, the extension of the pivot connections is pushed downward, causing a pivoting movement of the blades bearing the corresponding pivot connection.

Each set screw therefore parts two adjacent blades. Thus, the parting of the blades (2, 5) is obtained by acting on the set screw (210), the parting of the blades (2, 3) by acting on the set screw (310), the parting of the blades (3, 4) by acting on the set screw (410).

It is of course obvious that the screw device described hereinabove is given here by way of example and that any other means allowing the blades to be parted from one another could be used without departing from the scope of the present invention.

FIGS. 3 and 4 illustrate the successive positions conferred upon the blades (2 to 5) of the retractor (1) in order to move the retractor (1) from the contracted position of FIG. 2 into the parted position of FIG. 1.

In particular, FIG. 3 illustrates the parting of the pair of blades (2, 5) with the pair of blades (3, 4) of the retractor (1) that is obtained when the set screw (310) is operated first.

What happens is that when the set screw (310) is screwed into the corresponding retaining element (300), said set screw (310) exerts pressure on the extension of the pivot connection (20), thus causing the blade (2) to pivot with respect to the blade (3). As the blade (5) is not physically attached to the blade (4), the pivoting of the blade (2) with respect to the blade (3) causes the blade (5) to pivot with respect to the blade (4), the blades (5, 4) being fixed to the blades (2, 3) respectively.

FIG. 4 illustrates the parting of two other blades, when a further set screw is screwed in successively. In this instance, the set screw illustrated in FIG. 4 is the screw referenced (410). By screwing the set screw (410) into the corresponding retaining element (400), said screw (410) exerts pressure on the extension (31) of the pivot connection (30) thus causing the blade (4) to pivot with respect to the blade (3).

In order to obtain the optimum parted position of the retractor (1), which is the position illustrated in FIG. 1, all that is then required is for the remaining set screw (210) to be screwed into the corresponding retaining element (200), said set screw (210) exerting pressure on the extension (51) of the pivot connection (41) so as to cause the blade (5) to pivot with respect to the blade (2).

It is of course obvious that the present invention is not restricted to the order of screwing described above.

It should also be noted that the parting device described has the advantage not only of keeping the blades of the retractor (1) firmly held in a parted position, but also of adjusting the extent to which the blades (2 to 5) of said retractor (1) are parted.

Furthermore, from the foregoing description it is clearly evident that the blades (2 to 5) of the retractor (1) can be parted from one another independently.

Advantageously, said blades (2 to 5) are tile-shaped so that when the retractor (1) is in the contracted position, particularly while it is being inserted into the patient's tissues, the operating channel (6) formed by said blades (2 to 5) is of oval shape (cf. FIG. 2).

Advantageously, the blades (2 to 5) have a proximal end that is configured in such a way that, when they are connected to one another, the retractor (1) has a proximal part (7) of flared shape, which has the advantage of offering a good field of view of the operating channel (6).

With the blades (2 to 5) configured and arranged in this way, when the retractor (1) is in the contracted position it has more or less the shape of a funnel, the walls of this funnel consisting of said blades (2 to 5).

Advantageously, said blades (2 to 5) have, on their distal ends, teeth (9) extending transversely toward the tissues.

Advantageously, said blades (2 to 5) are made of stainless steel, aluminum, titanium or polymer.

In a preferred embodiment, said blades (2 to 5) will be made of radiotransparent material.

Likewise, according to an advantageous embodiment of the invention, said blades (2 to 5) are removable from the parting device. Thus, at any moment, it is possible to withdraw a blade, for example one which is too short, and replace it with a new, longer, blade, or vice versa. Likewise, it will be possible to replace each of the blades without it being necessary, once the retractor is positioned in the incision, to withdraw the parting device.

FIGS. 5 and 6 illustrate the retractor (1) equipped with a tubular protective membrane (10).

Said membrane (10), which is advantageously elastic, surrounds the blades of the retractor (1). It is held in place on the blades by means of notches (12) formed on the blades (2 to 5) (cf. FIG. 2).

Thus, when the retractor is in the parted position, the membrane makes it possible to prevent the retracted tissues from entering the spaces (11) formed between the blades (2 to 5). Thus, the retractor (1) equipped with the membrane (10) exhibits an operating channel that is free of any tissue. Furthermore, the membrane (10) allows bleeding into the operating channel (6) to be restricted. The thus-reduced amount of bleeding offers a better field of view of the operating channel (6), which is obviously of immediate benefit to the patient.

Advantageously, the membrane is made of tissue or of polymer of the polyurethane type.

According to one advantageous embodiment of the invention (and this embodiment is not depicted), the membrane (10) is fixed in a non-deployed state around the blades (2 to 5) of the retractor (1) when the retractor is in the contracted position and deploys along the blades (2 to 5) at the same time as they are parted.

The invention is described in the foregoing by way of example.-It must be understood that the person skilled in the art is able to vary the invention in various ways without thereby departing from the scope of the patent.

1. A retractor for retracting a patient's tissues, of the type comprising two blades each exhibiting a proximal end and a distal end, said blades being arranged in such a way as to form an operating channel open at the proximal ends and at the distal ends of said blades, wherein the retractor comprises at least one additional blade to form an at least three-bladed retractor, said blades being parted from one another by pivoting of their distal ends so as to form a widened operating channel of conical shape.

2. The retractor for retracting a patient's tissues as claimed in claim 1, wherein the retractor comprises a device for parting the blades which device consists of at least two bearing elements allowing a force to be exerted on the proximal end of said blades to cause said blades to part.

3. The retractor for retracting a patient's tissues as claimed in the preceding claim, wherein the bearing elements constitute elements for adjusting the extent of separation of said blades.

4. The retractor for retracting a patient's tissues as claimed in claim 2 or claim 3, wherein the bearing elements consist of screws, each of said screws passing through a threaded orifice formed in said blades.

5. The retractor for retracting a patient's tissues as claimed in any one of claims 1 to 4, wherein the retractor further comprises a tubular protective membrane surrounding said blades.

6. The retractor for retracting a patient's tissues as claimed in the preceding claim, wherein the blades have retaining notches for keeping the membrane fixed around said blades.

7. The retractor for retracting a patient's tissues as claimed in claim 5 or claim 6, wherein the membrane is elastic.

8. The retractor for retracting a patient's tissues as claimed in any one of claims 5 to 7, wherein the membrane is made of tissue or of polymer.

9. The retractor for retracting a patient's tissues as claimed in the preceding claim, wherein the membrane is made of polyurethane.

10. The retractor for retracting a patient's tissues as claimed in any one of claims 1 to 9, wherein the blades are removable.

11. The retractor for retracting a patient's tissues as claimed in any one of claims 1 to 10, wherein the blades are made of a radiotransparent material.

12. The retractor for retracting a patient's tissues as claimed in any one of claims 1 to 11, wherein the retractor comprises at least one access orifice providing access to the space situated between the tissues and the blades or, when the retractor comprises a protective membrane as claimed in claims 5 to 9, access to the space situated between the membrane and the blades surrounded by the said membrane.

13. The retractor for retracting a patient's tissues as claimed in any one of claims 1 to 12, wherein the retractor has a proximal part of flared shape.

14. The retractor for retracting a patient's tissues as claimed in any one of claims 1 to 13, wherein the retractor comprises means of attachment to a support.
